(19) United States
(12) Patent Application Publication
(10) Pub. No.: US 2013/0035390 A1
(43) Pub. Date: Feb. 7, 2013

(54) TREATMENT OF MULTIPLE SCLEROSIS

(71) Applicants: Yoel Kloog, Herzliya (IL); Elizabeta Aizman, Kiryat Motzkin (IL); Joab Chapman, Kiryat Ono (IL)

(72) Inventors: Yoel Kloog, Herzliya (IL); Elizabeta Aizman, Kiryat Motzkin (IL); Joab Chapman, Kiryat Ono (IL)

(73) Assignee: Ramot at Tel-Aviv University Ltd., Tel Aviv (IL)

(21) Appl. No.: 13/521,637

(22) PCT Filed: Jan. 13, 2011
(86) PCT No.: PCT/IB11/00213

§ 371 Date: Jul. 11, 2012

Related U.S. Application Data
(60) Provisional application No. 61/294,603, filed on Jan. 13, 2010.

(51) Int. Cl. A61K 31/197; A61P 25/00

(52) U.S. Cl. 514/564

(57) ABSTRACT

Disclosed are methods for treating multiple sclerosis patients that entail co-administration of effective amounts of a Ras antagonist which is farnesylthiosalicylic acid or an analog thereof, and a second active agent selected from glatiramer acetate, laquinimod and combinations thereof. Therapeutic compositions and methods of making them are also disclosed.

### CROSS-REFERENCE TO RELATED APPLICATIONS

This application claims the benefit of the filing date of U.S. Provisional Patent Application No. 61/294,603, filed Jan. 13, 2010, the disclosure of which is hereby incorporated herein by reference.

### BACKGROUND OF THE INVENTION

Multiple sclerosis (MS) is an inflammatory disease of the CNS affecting mainly white matter tracts. It affects mainly young people with a median age of onset at 30 years of age. The disease is chronic and runs over many years in a course that is first marked by acute exacerbations followed by remissions [McDonald, et al., Ann. Neurol. 50:121-127 (2001)]. The histological signs of disease include mainly inflammatory infiltration of the brain with lymphocytes and macrophages resulting in damage to myelin sheaths and axons [Noseworthy J H, et al., N. Engl. J. Med. 343:938-952 (2000); Trapp, et al., N. Engl. J. Med. 338:278-285 (1998)].

Each case of multiple sclerosis displays one of several patterns of presentation and subsequent course. Most commonly, multiple sclerosis first manifests itself as a series of attacks followed by complete or partial remissions as symptoms mysteriously lessen, only to return later after a period of stability. This is called relapsing-remitting (RR) multiple sclerosis. Primary-progressive (PP) multiple sclerosis is characterized by a gradual clinical decline with no distinct remissions, although there may be temporary plateaus or minor relief from symptoms. Secondary-progressive (SP) multiple sclerosis begins with a relapsing-remitting course followed by a later primary-progressive course. Rarely, patients may have a progressive-relapsing (PR) course in which the disease takes a progressive path punctuated by acute attacks. PP, SP, and PR are sometimes lumped together and called chronic progressive multiple sclerosis. A few patients experience malignant multiple sclerosis, defined as a swift and relentless decline resulting in significant disability or even death shortly after disease onset.

Many treatments have been tried for multiple sclerosis over the years, most of which affect the immune system. Interestingly, severe immunosupression has not been especially successful while more subtle approaches termed immunomodulation, which were the first evidence-based therapies to enter clinical practice, have been regarded as more effective [Goodin D S, et al., Subcommittee of the American Academy of Neurology and the Multiple Sclerosis Council for Clinical Practice Guidelines. Neurology 58:169-178 (2002)]. These therapies, however, have significant limitations in their effectiveness with some patients progressing in spite of optimal doses. Two main options have been proposed for the care of such patients with incomplete response to immunomodulatory drugs: The use of more extreme immunosuppressive approaches such as natalizumab, rituximab and cyclophosphamide [Menge T, et al., Drugs 68:2445-2468 (2008)] or the addition of relatively non-toxic drugs to the immunomodulatory ones [Hogh P, et al., Multiple Sclerosis 6:226-230 (2000)].

Accordingly, there is an existing need for effective therapies for multiple sclerosis.

### BRIEF SUMMARY OF THE INVENTION

One aspect of the present invention is directed to a method for treating a patient with multiple sclerosis. The method entails co-administering to the patient therapeutically effective amounts of a Ras antagonist which is farnesylthiosalicylic acid (also referred to herein as FTS or Salirasib) or an FTS analogue, which together are defined by the formula described herein, and a second active agent effective in the treatment of MS, selected from glatiramer acetate (also referred to herein as “GA”, “Copolymer 1” or Copaxone®), and laquinimod, and combinations thereof. In some embodiments, these active agents are administered in a single dosage form, which thus constitutes another aspect of the present invention. Compositions for use in practicing these methods, as well as methods of making them, are also provided.

A further aspect of the present invention is directed to a kit for use in treating multiple sclerosis, comprising a first dosage form containing therapeutically effective amounts of the Ras antagonist defined by the formula herein, and a second active agent effective in the treatment of MS, selected from glatiramer acetate and laquinimod and combinations thereof, or separate dosage forms containing the Ras antagonist and the second active agent, and optionally, written instructions for using the dosage form(s) to treat a multiple sclerosis patient. In certain embodiments, the Ras antagonist is FTS and is present in the kit in an oral formulation such as a tablet or capsule, and the glatiramer acetate is present in a solution for injection e.g., contained in a vial or a pre-filled syringe.

The present inventors have generated data showing that the combination of FTS and Copaxone® works synergistically in an acceptable animal model for multiple sclerosis.

### BRIEF DESCRIPTION OF THE DRAWINGS

FIGS. 1A and B are graphs illustrating that late treatment with FTS and GA suppresses the clinical signs of EAE. A. EAE was induced in C57bl/6 mice with MOG in CFA and pertussis. Animals were treated daily, starting from day 9 following EAE-induction, FTS together with GA, or with each on of them separately or the vehicle (n=30 per each group). The severity of EAE was graded according to a 0-6 scale (as described in Materials and methods). The graph shows the mean clinical scores per group daily. ***P<0.001, Kruskal-Wallis test. B. Animals were treated daily, starting from day 9 following EAE-induction, either with i.p. injections of FTS (20 mg/kg/day) together with s.c. injections of GA (15 mg/kg/day), or with each one of them separately or the vehicle (n=40 per each group). ***P<0.01, ***P<0.001, Kruskal-Wallis test.

FIG. 2 collectively shows that combined treatment of FTS and GA reduces the MRI lesions and disruption of the blood-brain barrier in the spinal cords of EAE mice. EAE mice were treated daily, starting from day 9 following EAE-induction, either with i.p. injections of FTS (20 mg/kg/day) together with s.c. injections of GA (15 mg/kg/day), or with each one of them separately or the vehicle. On day 14 post EAE-induction, the lower limb plexuses (L1-S3) of the mice's spinal cord was scanned using an MRI system (n=8 per group). A. T₂-map images (TR 3600 ms, TE 16 ms) were sequenced. Representative images are presented. B. The analysis of T₂-map MRI was performed by selecting ROIs corresponding to lesion and parallel normal area in the same slice. The sum of T₂ value of the enhancing region in each slice (20 slices per mouse) was multiplied by the number of voxels in that region and then divided by the sum of voxels per mouse. From that value was then subtracted the value of a normal parallel tissue which was measured in the same way as the enhancing region (as described in Materials and methods). ***P<0.001, Kruskal-Wallis test. C. The total volume (in mm³) of T₂-map enhanced regions was defined and accumulated. **P<0.01, Kruskal-Wallis test. D. T₁-weighted images (TR 1100 ms, TE 9.754 ms) were sequenced before and after administration of 0.5 mmol/kg body weight Gd-DTPA. Gadolimium enhanced regions were defined and their volume (in mm³) was accumulated. Representative images are presented in the upper panel. E. Statistical analysis of the results is presented in the lower panel wherein the total volume (in mm³) of T₁-map enhanced regions was defined and accumulated. ***P<0.001, Kruskal-Wallis test.

FIG. 3 collectively shows that combined treatment of FTS with GA reduces the infiltration and demyelination in the spinal cords of EAE mice. EAE mice were treated daily, starting from day 9 following EAE-induction, either with i.p. injections of FTS (20 mg/kg/day) together with s.c. injections of GA (15 mg/kg/day), with each one of them separately or with the vehicle. On day 16 post EAE-induction, animals were sacrificed and their lumbar part of the spinal cord were fixed and embedded in paraffin as described in Material and methods. The Spinal cord sections were prepared and stained with H&E and LFB (n=8 per each group). A. Representative sections of the spinal cord from each group stained with H&E. Scale bar 500 μm for the upper panel and 200 μm for the lower panel. B. A quantification plot depicting the percentage of cell counts per field which were conducted using Image-Pro Plus software. *P<0.05, Kruskal-Wallis test. C. Representative sections of the spinal cord from each group stained with LFB. Scale bar 500 μm for the upper panel and 200 μm for the lower panel. D. A quantification plot depicting the percentage of regions of demyelination per field conducted using the Image-Pro software. **P<0.01, Kruskal-Wallis test.

FIG. 4 collectively shows that combined treatment of FTS and GA in vivo induces the amount of Foxp3 and reduces the amount of Ras, Ras-GTP and P-Erk in the splenocytes and the amount of lymphocytes in the brain. A. EAE mice were treated daily, starting from day 9 following EAE-induction, either with i.p. injections of FTS (20 mg/kg/day) together with s.c. injections of GA (15 mg/kg/day), or with each one of them separately or the vehicle. Foxp3, Ras, Ras-GTP, Erk, P-Erk and 13-tubulin levels in splenocytes lysates were assayed by western blotting as described in example I. Upper panel: Representative blots. Lower panel: densitometry analysis of Foxp3(B), Ras(C), Ras-GTP(D) and P-Erk(E). *P<0.05, ANOVA. F. CD3, Foxp3 and β-tubulin levels in the mice's brains were assayed by western blotting. Upper panel: Representative blots. Lower panel: densitometry analysis of CD3(G) and Foxp3(H). *P<0.05, ANOVA.

FIG. 5. Ex vivo proliferation response of EAE splenocytes to various antigens and analysis of serum cytokines. Animals were immunized for induction of EAE. A. Lymphocytes were obtained from the spleens of FTS plus GA, FTS, GA or vehicle-treated animals, on day 16 post EAE-induction and then cultured ex vivo in the presence of various mitogens and myelin antigen (MOG) (n=8 per group) as follows: 25 μg/ml MOG, 50 μg/ml MOG, 20 μg/ml LPS and 1 μg/ml ConA. A bromodeoxyuridine (BrdU) incorporation assay was performed and the BrdU incorporation values of the absorbance at 450 nm in the presence of the antigen/absorbance at 450 nm without the antigen (S.I.) are given (n=8 per group. S.I.=stimulation index) as described in Materials and methods section. **P<0.01 vs. control group, Kruskal-Wallis test. B-D. Serum was obtained from FTS plus GA−, FTS−, GA− or vehicle-treated animals (n=10 per each group), on day 16 post EAE-induction, and the levels of four cytokines IL-10 (B), IL-4 (C), IFN-γ (D) and IL-17 (E) were detected. The values of cytokine levels are represented in pg/ml. *P<0.05, **P<0.01, ***P<0.001 vs. control group or vs. other group as indicated, Kruskal-Wallis test.

FIG. 6. A proposed model explaining the synergistic attenuates of EAE by combined treatment of GA and FTS: Two distinct mechanisms prevent autoimmunity. Differentiation and maturation of DC are mediated through different stimuli. Whereas 1,25-dihydroxyvitamin D3, IL-10 and vasoactive intestinal peptide (VIP-1) induce the differentiation towards tolerogenic DCs (Auray, et al.; Chorny, et al., 2005; Wakkach, et al., 2003), bacterial and viral antigens (LPS, CpG) result in their maturation, thereby induce naïve T cell differentiation into effectors Th1 and Th17 (Arm 1) (Reis e Sousa, 2006). The differentiated effector T cells secrete pro-inflammatory cytokines (TNF-α, IFN-γ) which induce neuroimmunity (EAE) (Bertolotto, et al., 1999; Killestein, et al., 2001). The tolerogenic DCs have a crucial role in the maintenance of immune tolerance. First, they upregulate CD4⁺CD25⁺Foxp3⁺ T regulatory cells (Treg) which in turn inhibit the proliferation of effector T cells and maintain immune tolerance (Schildknecht, et al.). Previous studies demonstrated a direct influence of the Ras Cascade on regulatory T cells (Li, et al., 2005; Mor, et al., 2008, 2009). It was shown that Ras inhibition by FTS augments Foxp3⁺ Tregs, indicating that FTS blocks Ras-GTP and the MAPK cascade, thereby relieving the inhibition of Foxp3 expression (Arm 3) (Mor, et al., 2008, 2009). An additional pathway to induce tolerance by DCs includes differentiation towards Th2 cells that leads to the secretion of inflammatory cytokines (IL-10 and TGF-β) (Arm 2) (Kalinski, et al., 1999). Both MOG and GA are presented by DCs on their MHC-II receptor (Ben-Nun, et al., 1996). Whereas MOG results in differentiation towards mature DCs and immunity (Isaksson, et al., 2009), GA induces the differentiation of tolerogenic DCs which induce Th2 cells (Vieira, et al., 2003). The outstanding phenomena of the combination of tolerance enhancement (by GA) and relieving from Ras-dependant tolerance inhibitor (FTS) lead to the strong synergistic effect which reduced EAE symptoms.

FIG. 7. Late treatment with FTS per os or subcutaneous together with GA suppresses the clinical signs of EAE. A. EAE was induced in C57bl/6 mice with MOG in CFA and pertussis. Animals were treated daily, starting from day 9 following EAE-induction, FTS (60 mg/kg/mouse, p.o.) together with GA (15 mg/kg/mouse, s.c.), or with each one of them separately or the vehicle (n=10 per group). The severity of EAE was graded according to a 0-6 scale (as described in Materials and Methods). The graph shows the mean clinical scores per group daily. ***P<0.001, Kruskal-Wallis test. B. Animals were treated daily, starting from day 9 following EAE-induction, either with s.c. injections of FTS (40 mg/kg/day) dissolved in GA (15 mg/kg/day), or with each one of the separately or the vehicle (n=10 per each group). ***P<0.01, Kruskal-Wallis test.

### DETAILED DESCRIPTION

Patients having multiple sclerosis may be identified in accordance with diagnostic protocols known in the art. For example, multiple sclerosis patients may be identified by criteria establishing a diagnosis of clinically definite multiple sclerosis (Poser, et al., Ann. Neurol. 13:227, 1983). Briefly, an individual with clinically definite multiple sclerosis has had two attacks and clinical evidence of either two lesions or clinical evidence of one lesion and paraclinical evidence of another, separate lesion. Definite multiple sclerosis may also be diagnosed by evidence of two attacks and oligoclonal bands of IgG in cerebrospinal fluid or by combination of an attack, clinical evidence of two lesions and oligoclonal band of IgG in cerebrospinal fluid. The McDonald criteria can also be used to diagnose multiple sclerosis. (McDonald, et al., 2001, Ann Neurol 50:121-127). The McDonald criteria include the use of MRI evidence of CNS impairment over time to be used in diagnosis of multiple sclerosis, in the absence of multiple clinical attacks.

### The Ras Antagonists

Ras proteins e.g., H—, N— and K-Ras, act as on-off switches that regulate signal-transduction pathways controlling cell growth, differentiation, and survival. [Reuther, et al., Curr. Opin. Cell Biol. 12:157-65 (2000)]. They are anchored to the inner leaflet of the plasma membrane, where activation of cell-surface receptors, such as receptor tyrosine kinase, induces the exchange of guanosine diphosphate (GDP) for guanosine triphosphate (GTP) on Ras and the conversion of inactive Ras-GDP to active Ras-GTP. [Scheffzek, et al., Science 277:333-7 (1997)]. Termination of these signals involves hydrolysis of the Ras-GTP to Ras-GDP. [Scheffzek, et al., Science 277:333-338 (1997).] Besides the cell-proliferation promotion by wild-type Ras, several mutated forms of Ras are defective in their GTP hydrolysis liability and are therefore constitutively active. [Barbacid, Biochem. 56:779-827 (1987); Box, Eur. J. Cancer 31:1051-1054 (1995).] These oncogenic Ras proteins, which are found in many cancer types, contribute to malignancy and are therefore considered favored targets for directed therapy. [Bos, Cancer Res. 49:4682-4689 (1989).] The active Ras protein promotes oncogenesis through activation of multiple Ras effectors that contribute to deregulated cell growth, differentiation, and increased survival, migration and invasion. [See, e.g., Downward, Nat. Rev. Cancer 3:11-22 (2003); Shields, et al., Trends Cell Biol. 10:147-541 (2000); and Mitin, et al., Curr. Biol. 15:R563-74 (2005).]

Association of Ras to the plasma membrane has been shown to be crucial for its activity in both the wild-type and the mutated constitutively active forms [Boguski, et al., Nature, 366:643-654 (1993); Cox, et al., Curr. Opin. Cell Biol. 4:1008-1016 (1992); Marshall, Curr. Opin. Cell Biol. 8:197-204 (1996)]. At least two structural elements are required for this association; the first is a farnesylcysteine carboxy methyl ester at the carboxy terminal of Ras, and the second element resides at the adjacent upstream sequence and varies among different Ras isoforms. [Hancock, et al., EMBO J. 10:4033-4039 (1991); Hancock, et al., Cell 57:1167-1177 (1989)] Normal Ras activity specifically requires the farnesyl isoprenoid moiety [Cox, et al., Curr. Opin. Cell Biol. 4:1008-1016 (1992); Cox, et al., Mol. Cell. Biol. 12:2606-2615 (1992)] which acts as a specific recognition unit to allow binding of H-Ras with galectin-1 [Elad-Sfadia, et al., J. Biol. Chem. 277:37169-37175 (2002), Rotblat, et al., J. Biol. Chem. 64:3112-3118 (2004)] and K-Ras with galectin-3 [Elad-Sfadia, et al., J. Biol. Chem. 279:34922-34930 (2004)], promoting strong membrane association and robust signaling.

FTS is known as a Ras inhibitor that acts in a rather specific manner on the active, GTP-bound forms of H-, N-, and K-Ras proteins. [Weisz, et al., Oncogene 18:2579-2588 (1999); Gana-Weisz, et al., Clin. Cancer Res. 8:555-65 (2002)]. More specifically, FTS competes with Ras-GTP for binding to specific saturable binding sites in the plasma membrane, resulting in mislocalization of active Ras and facilitating Ras degradation. [Haklai, et al., Biochemistry 37(5):1306-14 (1998)]. This competitive inhibition prevents active Ras from interacting with its prominent downstream effectors and results in reversal of the transformed phenotype in transformed cells that harbor activated Ras. As a consequence, Ras-dependent cell growth and transforming activities, both in vitro and in vivo, are strongly inhibited by FTS. [Weisz, et al., supra.; Gana-Weisz, et al., supra.].

Ras antagonists useful in the present invention include FTS and its structural analogs, are described below.

The Ras antagonists are represented by the formula:

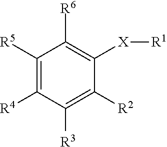

wherein X represents S; wherein R¹ represents farnesyl, or geranyl-geranyl; R² is COOR⁷, CONR⁷R⁸, or COOCHR⁹OR¹⁰, wherein R⁷ and R⁸ are each independently hydrogen, alkyl, or alkenyl, including linear and branched alkyl or alkenyl, which in some embodiments includes C₁-C₄ alkyl or alkenyl; wherein R⁹ represents H or alkyl; and wherein R¹⁰ represents alkyl, including linear and branched alkyl and which in some embodiments represents C₁-C₄ alkyl; and wherein R³, R⁴, R⁵ and R⁶ are each independently hydrogen, alkyl, alkenyl, alkoxy (including linear and branched alkyl, alkenyl or alkoxy and which in some embodiments represents C₁-C₄ alkyl, alkenyl or alkoxy), halo, trifluoromethyl, trifluoromethoxy, or alkylmercapto. In embodiments wherein any of R⁷, R⁸, R⁹ and R¹⁰ represents alkyl, it is preferably methyl or ethyl.

In some embodiments, the Ras antagonist is S-trans,trans-farnesylthiosalicylic acid or FTS (wherein R¹ is farnesyl, R² is COOR⁷, and R⁷ is hydrogen).

In some embodiments, the FTS analog is halogenated, e.g., 5-chloro-FTS (wherein R¹ is farnesyl, R² is COOR⁷, R⁴ is chloro, and R⁷ is hydrogen), and 5-fluoro-FTS (wherein R¹ is farnesyl, R² is COOR⁷, R⁴ is fluoro, and R⁷ is hydrogen).

In other embodiments, the FTS analog is FTS-methyl ester (wherein R¹ represents farnesyl, R² represents COOR⁷, and R⁷ represents methyl), FTS-amide (wherein R¹ represents farnesyl, R² represents CONR⁷R⁸, and R⁷ and R⁸ both represent hydrogen); FTS-methylamide (wherein R¹ represents farnesyl, R² represents CONR⁷R⁸, R⁷ represents hydrogen and R⁸ represents methyl); and FTS-dimethylamide (wherein R¹ represents farnesyl, R² represents CONR⁷R⁸, and R⁷ and R⁸ each represents methyl).

In yet other embodiments, the Ras antagonist is an alkoxyalkyl S-prenylthiosalicylate or an FTS-alkoxyalkyl ester (wherein R² represents COOCHR⁹OR¹⁰). Representative examples include methoxymethyl S-farnesylthiosalicylate (wherein R¹ is farnesyl, R⁹ is H, and R¹⁰ is methyl); methoxymethyl S-geranylgeranylthiosalicylate (wherein R¹ is geranylgeranyl, R⁹ is H, and R¹⁰ is methyl); methoxymethyl 5-fluoro-5-farnesylthiosalicylate (wherein R¹ is farnesyl, R⁵ is fluoro, R⁹ is H, and R¹⁰ is methyl); and ethoxymethyl S-farnesylthiosalicyate (wherein R¹ is farnesyl, R⁹ is methyl and R¹⁰ is ethyl). In each of the embodiments described above, unless otherwise specifically indicated, each of R³, R⁴, R⁵ and R⁶ represents hydrogen.

### Copaxone®

Copaxone® is the brand name for glatiramer acetate (also known as Copolymer 1). Glatiramer acetate (GA), the active ingredient of Copaxone®, contains the acetate salts of synthetic polypeptides, containing four naturally occurring amino acids: L-glutamic acid, L-alanine, L-tyrosine, and L-lysine with average molar fractions of [L-Glu: 0.129-0.153; L-Ala: 0.392-0.462; L-Tyr: 0.086-0.100; L-Lys: 0.300-0.374] respectively. The average molecular weight of glatiramer acetate is 4,700-11,000 daltons. Chemically, glatiramer acetate is designated L-glutamic acid polymer with L-alanine, L-lysine and L-tyrosine, acetate (salt). Its structural formula is described in “Copaxone”, Physician's Desk Reference, (2000), Medical Economics Co., Inc., (Montvale, N.J.), at 3115. Glatiramer acetate is also written as: poly [L-Glu¹³⁻¹⁵, L-Ala³⁹⁻⁴⁶, L-Tyr^{8.6-10}, L-Lys³⁰⁻³⁷]nCH₃COOH.

The mechanisms by which glatiramer acetate ameliorates multiple sclerosis are not fully elucidated, but some important immunological aspects of these features have been studied and reported in the literature. For example, GA shows some cross reactivity with Myelin Basic Protein (MBP), mediated by both T-cells and antibodies. It binds to various Major Histocompatibility Complex (MHC) class II molecules on antigen-presenting cells (APC) and prevents them from binding to T-cells with several antigen-recognition properties (Fridkis-Hareli, M., et al., Proc. Natl. Acad. Sci. (USA), 1994, 91:4872-4876. In rodents, GA suppresses the encephalitogenic effects of auto reactive T-cells. Passive transfer of GA-reactive T-cells prevents the development of EAE induced in rats or mice by MBP, protolipid protein (PLP) or Myelin Oligodendrocyte Glycoprotein (MOG) (Aharoni, D., et al., Eur. J. Immunol., 1993, 23:17-25). In humans, daily injection of GA, resulted in the development of a T helper-2 (Th2)-type of protective response over time. These activated GA-reactive T-cells, when reaching the site of injury, secrete cytokines associated with both Th2 (IL-4) profiles and neurotrophic factors such as Brain Derived Neurotrophic Factor (Ziemessen) (BDNF), and thus serve a dual role: first exerting bystander suppression anti-inflammatory activity and later a neuroprotective action on axons.

Thus, GA is believed to have a dual mechanism of action. As an immunomodulating agent, it stimulates Th2 cells to secrete both anti-inflammatory cytokines as well as BDNF. This provides an anti-inflammatory milieu and neurotrophic support to the demyelinating axons protecting them from further degeneration over the long term. These features of GA are reflected in both the long-term efficacy of GA in reducing relapse rate as well as in affecting Magnetic Resonance Imaging (MRI) markers of axonal loss. Comi G., et al., Annals of Neurology; 44(3):507, 1998; Comi G., et al., Neurology; 52(6) Suppl. 2:A263-265, A289-A291, A336, A464, A491-A494, A496-A499, 1999). In this study, significantly fewer gadolinium-enhancing lesions progressed to persistent black holes in the GA-treated group than in the group receiving placebo. This suggests that GA may have the capacity to offer an axonal protective effect (Filippi, et al., Neurol., 57:731-733, 2001).

Methods of making GA are known in the art. For example, U.S. Pat. No. 3,849,550 teaches a process in which the N-carboxyanhydrides of tyrosine, alanine, γ-benzyl glutamate and ε-N-trifluoro-acetyl lysine are polymerized in anhydrous dioxane with diethylamine as initiator. The deblocking of the γ-carboxyl group of the glutamic acid is effected by hydrogen bromide in glacial acetic acid and is followed by the removal of the trifluoroacetyl groups from the lysine residues by 1 M piperidine. Another process for making Copaxone® is described in U.S. Patent Application Publication 20070141663.

### Laquinimod

Laquinimod is a quinoline derivative. It is the sodium salt of 5-chloro-N-ethyl-4-hydroxy-1-methyl-2-oxo-N-phenyl-1,2-dihydroquinoline-3-carboxamide.

Methods of making laquinimod are known in the art. See, e.g., U.S. Pat. Nos. 6,077,851; 6,875,869; 7,560,557; and 7,589,208.

### Compositions and Methods

The terms “administer”, “administering”, “administration”, and the like, as used herein, refer to the methods that may be used to enable delivery of compounds or compositions to the desired site of biological action. Medically acceptable administration techniques suitable for use in the present invention are known in the art. See, e.g., Goodman and Gilman, The Pharmacological Basis of Therapeutics, current ed.; Pergamon; and Remington's, Pharmaceutical Sciences (current edition), Mack Publishing Co., Easton, Pa. In some embodiments, at least one or both the Ras antagonist and the second active agent are administered orally. In other embodiments, at least one or both the Ras antagonist and the second active agent are administered parenterally (which for purposes of the present invention, includes intravenous, subcutaneous, intraperitoneal, intramuscular, intravascular and infusion).

The Ras antagonist and the second active agent are co-administered, which as used herein, encompasses treatment regimens in which these agents are administered to the multiple sclerosis patient at the same or different times (i.e., substantially simultaneously or sequentially), and by the same or different route of administration, such that both agents and/or their metabolites are present in the patient at the same time in order to achieve the benefits of their combined therapeutic effect. Co-administration thus includes simultaneous administration in separate compositions, administration at different times in separate compositions, and/or administration in a composition that contains both agents. In preferred embodiments, the Ras antagonist, e.g., FTS, is administered orally or subcutaneously, and the second active agent is administered subcutaneously. In some embodiments, the Ras antagonist is administered by dosing orally on a daily basis (in single or divided doses) for three weeks, followed by a one-week “off period”, and repeating until remission is achieved. In these embodiments, the second active agent may be present in the same composition, e.g., wherein the Ras antagonist and laquinimod are in the same oral dosage form such as a tablet, or in the same dosage form formulated for s.c. administration. In some other embodiments, GA is administered daily via s.c. administration, in single or divided dosages (e.g., 2 or 3 times daily).

The term “therapeutically effective amounts”, as used herein, refers to a sufficient amount of each of the Ras antagonist and the second active agent that will ameliorate at least one symptom of the multiple sclerosis and its associated manifestations, diminish the extent or severity of the disease, delay or retard disease progression, achieve partial or complete remission, prolong survival and combinations thereof. As shown in the working examples, combinations of the Ras antagonist and GA achieve synergy, i.e., a greater than additive effect. Applicants believe that these results reflect decreased disease activity in vivo, and ultimately result in more effective multiple sclerosis therapy and a commensurate improvement in one or more of these clinical manifestations of the disease, as described below.

Effective treatment of multiple sclerosis may be evaluated in several different ways. For example, the following parameters can be used to gauge effectiveness of treatment. Three exemplary criteria include: EDSS (extended disability status scale), appearance of new lesions on MRI (magnetic resonance imaging), and clinical exacerabations. The EDSS is a means to grade clinical impairment due to multiple sclerosis (Kurtzke, Neurology 33:1444, 1983). Functional systems that may be evaluated prior to treatment for the type and severity of neurologic impairment include pyramidal, cerebella, brainstem, sensory, bowel and bladder, visual and cerebral. Follow-ups may be conducted at defined intervals. The scale ranges from 0 (normal) to 10 (death due to multiple sclerosis). A decrease of one full step indicates an effective treatment (Kurtzke, Ann. Neurol. 36:573-79 1994).

Clinical exacerbations include the appearance of a new symptom that is attributable to multiple sclerosis and accompanied by an appropriate new neurologic abnormality. Exacerbations may be either mild, moderate, or severe, and may be graded according to changes in a Neurological Rating Scale (Sipe, et al., Neurology 34:1368, 1984). An annual exacerbation rate and proportion of exacerbation-free patients may be determined.

Likewise, methods for assessing whether therapy is effective are known in the art. For example, therapy may be deemed to be effective if there is a statistically significant difference in the rate or proportion of exacerbation-free or relapse-free patients between the treated group and the placebo group for either of these measurements. In addition, time to first exacerbation and exacerbation duration and severity may also be measured. A measure of effectiveness as therapy in this regard is a statistically significant difference in the time to first exacerbation or duration and severity in the treated group compared to control group. An exacerbation-free or relapse-free period of greater than one year, 18 months, or 20 months is particularly good evidence of effective therapy.

Clinical measurements include the relapse rate in one and two-year intervals, and a change in EDSS, including time to progression from baseline of 1.0 unit on the EDSS that persists for six months. On a Kaplan-Meier curve, a delay in sustained progression of disability shows efficacy. Other criteria include a change in area and volume of T₂ images on MRI, and the number and volume of lesions determined by gadolinium-enhanced images.

MRI can be used to measure active lesions using gadolinium-DTPA-enhanced imaging (McDonald, et al., Ann. Neurol. 36:14, 1994) or the location and extent of lesions using T₂-weighted techniques. Briefly, baseline MRIs are obtained. The same imaging plane and patient position are used for each subsequent study. Positioning and imaging sequences can be chosen to maximize lesion detection and facilitate lesion tracing. The same positioning and imaging sequences can be used on subsequent studies. The presence, location and extent of multiple sclerosis lesions can be determined by radiologists. Areas of lesions can be outlined and summed slice-by-slice for total lesion area. Three analyses may be done, namely: evidence of new lesions; rate of appearance of active lesions; and percentage change in lesion area (Paty, et al., Neurology 43:665, 1993). Improvement due to therapy can be established by a statistically significant improvement in an individual patient compared to baseline or in a treated group versus a placebo group.

Methods of the present invention may be effective in ameliorating at least one symptom associated with multiple sclerosis, includes optic neuritis, diplopia, nystagmus, ocular dysmetria, internuclear opthalmoplegia, movement and sound phosphenes, afferent pupillary defect, paresis, monoparesis, paraparesis, hemiparesis, quadraparesis, plegia, paraplegia, hemiplegia, tetraplegia, quadraplegia, spasticity, dysarthria, muscle atrophy, spasms, cramps, hypotonia, clonus, myoclonus, myokymia, restless leg syndrome, footdrop, dysfunctional reflexes, paraesthesia, anaesthesia, neuralgia, neuropathic and neurogenic pain, l'hermitte's, proprioceptive dysfunction, trigeminal neuralgia, ataxia, intention tremor, dysmetria, vestibular ataxia, vertigo, speech ataxia, dystonia, dysdiadochokinesia, frequent micturation, bladder spasticity, flaccid bladder, detrusor-sphincter dyssynergia, erectile dysfunction, anorgasmy, frigidity, constipation, fecal urgency, fecal incontinence, depression, cognitive dysfunction, dementia, mood swings, emotional lability, euphoria, bipolar syndrome, anxiety, aphasia, dysphasia, fatigue, Uhthoff's symptom, gastroesophageal reflux, and sleeping disorders.

The average daily dose of the Ras antagonists of the present invention generally ranges from about 200 mg to about 2000 mg, in some embodiments from about 400 to about 1600 mg, and in some other embodiments from about 600 to about 1200 mg, and in yet other embodiments, from about 400 mg to about 1200 mg, or from about 800 mg to about 1200 mg. These ranges include oral and parenteral administration.

Subcutaneous (s.c.) administration of Copaxone® is preferred. Daily dosage ranges for s.c. administration generally range from about 5 mg/day to about 25 mg/day, and in some embodiments from about 10 mg/day to about 20 mg/day, and in preferred embodiments about 20 mg/day. The recommended dosing schedule of Copaxone® for relapsing-remitting, multiple sclerosis is 20 mg/day injected subcutaneously (Physician's Desk Reference, 2003; see also U.S. Pat. Nos. 3,849,550; 5,800,808; 5,858,964, 5,981,589; 6,048,898; 6,054,430; 6,214,791; 6,342,476; 6,362,161; 6,620,847; 6,939,539; and 7,199,028. Oral formulations and appropriate dosage amounts are also known in the art. See, e.g., U.S. Patent Application Publication 20010055568; and U.S. Patent Application Publication 20010055568 (teaching oral formulations of Copaxone® with microcrystalline cellulose).

Daily doses of laquinimod for use in the treatment of MS generally range from about 0.0005 mg/kg to about 10 mg/kg body weight, in some embodiments from about 0.005 mg/kg to 1 mg/kg body weight. In some other embodiments, laquinimod is administered in a flat daily dosage of about 0.1 mg to about 1.5 mg (and in yet other embodiments a daily dosage of about 0.6 mg).

The term “pharmaceutically acceptable” as used herein, refers to a material, such as a carrier and other non-active excipients, which does not abrogate the biological activity or properties of the active agent(s), and is relatively nontoxic.

The term “pharmaceutical composition,” as used herein, refers to the Ras antagonist and/or the second active agent, optionally combined (e.g., mixed) with a pharmaceutically acceptable carrier. These ingredients are non-toxic, physiologically inert and do not adversely interact with the active agent(s) present in the composition. Carriers facilitate formulation and/or administration of the active agents. Pharmaceutical compositions of the present invention may further contain one or more excipients.

Oral compositions for the Ras antagonist and/or the second active agent can be prepared by bringing the agent(s) into association with (e.g., mixing with) the carrier, the selection of which is based on the mode of administration. Carriers are generally solid or liquid. In some cases, compositions may contain solid and liquid carriers. Compositions suitable for oral administration that contain the active are preferably in solid dosage forms such as tablets (e.g., including film-coated, sugar-coated, controlled or sustained release), capsules, e.g., hard gelatin capsules (including controlled or sustained release) and soft gelatin capsules, powders and granules. The compositions, however, may be contained in other carriers that enable administration to a patient in other oral forms, e.g., a liquid or gel. Regardless of the form, the composition is divided into individual or combined doses containing predetermined quantities of the active ingredient or ingredients.

Oral dosage forms may be prepared by mixing the active pharmaceutical ingredient or ingredients with one or more appropriate carriers (optionally with one or more other pharmaceutically acceptable excipients), and then formulating the composition into the desired dosage form e.g., compressing the composition into a tablet or filling the composition into a capsule or a pouch. Typical carriers and excipients include bulking agents or diluents, binders (e.g., polyvinylpyrrolidone, starch and hydroxypropyl methylcellulose), buffers or pH adjusting agents, disintegrants (including crosslinked and super disintegrants such as croscarmellose), glidants, and/or lubricants, including lactose, starch, mannitol, microcrystalline cellulose, ethylcellulose, sodium carboxymethylcellulose, hydroxypropylmethylcellulose, calcium sulfate, calcium hydrogen phosphate, dibasic calcium phosphate, acacia, gelatin, stearic acid, magnesium stearate, corn oil, vegetable oils, and polyethylene glycols. Coating agents such as sugar, shellac, and synthetic polymers may be employed, as well as colorants and preservatives. See, Remington's Pharmaceutical Sciences, The Science and Practice of Pharmacy, 20th Edition (2000). A purportedly stability-enhanced solid dosage form of laquinimod, which is disclosed in U.S. Pat. No. 7,589,208, includes, in addition to laquinimod, an alkaline-reacting component (e.g., sodium, potassium, calcium and aluminum salts of acetic acid, carbonic acid, citric acid or phosphoric acid) or a salt with a divalent metal cation (e.g., calcium acetate), and a pharmaceutical excipient.

Liquid form compositions include, for example, solutions, suspensions, emulsions, syrups, elixirs and pressurized compositions. The active agent(s), for example, can be dissolved or suspended in a pharmaceutically acceptable liquid carrier such as water, an organic solvent (and mixtures thereof), and/or pharmaceutically acceptable oils or fats. Examples of liquid carriers for oral administration include water (particularly containing additives as above, e.g., cellulose derivatives, preferably in suspension in sodium carboxymethyl cellulose solution), alcohols (including monohydric alcohols (including monohydric alcohols and polyhydric alcohols, e.g., glycerin and non-toxic glycols) and their derivatives, and oils (e.g., fractionated coconut oil and arachis oil). The liquid composition can contain other suitable pharmaceutical additives such as solubilizers, emulsifiers, buffers, preservatives, sweeteners, flavoring agents, suspending agents, thickening agents, colorants, viscosity regulators, stabilizers or osmoregulators.

Carriers suitable for preparation of compositions for parenteral administration include aqueous solutions such as Sterile Water for Injection, Bacteriostatic Water for Injection, Sodium Chloride Injection (0.45%, 0.9%), Dextrose Injection (2.5%, 5%, 10%), Lactated Ringer's Injection, and the like. Dispersions can also be prepared in glycerol, liquid polyethylene glycols and mixtures thereof, and in oils. Compositions may also contain tonicity agents (e.g., sodium chloride and mannitol), antioxidants (e.g., sodium bisulfite, sodium metabisulfite and ascorbic acid) and preservatives (e.g., benzyl alcohol, methyl paraben, propyl paraben and combinations of methyl and propyl parabens).

In preferred embodiments, the Ras antagonist, e.g., FTS, is formulated in a tablet (e.g., with microcrystalline cellulose) or in a soft gelatin capsule, in a dosage amount of about 200 to about 300 mg, and in some embodiments about 200, about 250 or about 300 mg. In some preferred embodiments, for example, FTS is formulated in a tablet in an amount of about 200 mg, with microcrystalline cellulose (e.g., about 210 mg), hydroxypropylmethyl cellulose (also known as hypromellose) (e.g., about 12 mg), croscarmellose sodium as disintegrant (e.g., about 18 mg) and magnesium stearate as lubricant (e.g., about 4 mg). In other preferred embodiments, laquinimod is also present, in an amount ranging from about 0.1 mg_ to about 1.5 mg.

In preferred embodiments that involve use of GA, the glatiramer acetate is formulated in a solution for subcutaneous injection containing water (e.g., about 1 ml), mannitol (e.g., about 40 mg), in an amount of about 20 mg.

The pharmaceutical composition containing the Ras antagonist and the second active agent, or first and second compositions containing the Ras antagonist and the second active agent respectively, may be packaged and sold in the form of a kit. For example, the kit may contain one or more oral dosage forms of the Ras antagonist, e.g., FTS, such as tablets or capsules (e.g., hard or soft gelatin capsules), and one or more s.c. dosage forms of glatiramer acetate contained in a vial or pre-filled syringe. In other embodiments, the kit may contain one or more oral dosage forms of the Ras antagonist, e.g., FTS, such as tablets or capsules (e.g., hard or soft gelatin capsules), and one or more oral dosage forms of laquinimod (e.g., capsules or tablets). In yet other embodiments, the kit may contain one or more oral dosage forms such as a tablet or capsule (e.g., hard or soft gelcap) that contains both the Ras antagonist, e.g., FTS, and laquinimod. The kit may also contain written instructions for carrying out the inventive methods as described herein.

The present invention will now be described by way of the following non-limiting examples. Unless otherwise stated, all parts are by weight.

### Example I

### A Combined Treatment of Copaxone® and Salirasib Resulted in a Complete Block of Experimental Allergic Encephalomyelitis (EAE) in Mice.

The animal model widely found useful for multiple sclerosis research is experimental autoimmune encephalomyelitis (EAE). Active immunization with myelin or component peptides or passive transfer of myelin-reactive lymphocytes causes inflammation relatively specific for white matter together with clinical features compatible with multiple sclerosis. The experimental work described in this example involved the combined effect of salirasib and GA on the EAE model. The results obtained from these experiments indicate a significant synergistic effect of the combined therapy which indicates clinical usefulness.

### Materials and Methods

### Mice

Eight-week-old female C57bl/6 mice were purchased from Harlan. The mice were housed under standard conditions in top filtered cages. Mice were fed a regular diet and given acidified water without antibiotics.

### Induction and Evaluation of EAE

Disease was induced by immunization with the peptide encompassing amino acids 35-55 of rat MOG synthesis (Anaspec, Fremont, Calif.). Mice were injected subcutaneously (s.c.) at the flank, with a 200 μl emulsion containing 300 μg of MOG in complete Freund's adjuvant (CFA) enriched with 500 μg Mycobacterium tuberculosis H37Ra (Difco Laboratories, Detroit, Mich.). Pertussis toxin (List biological laboratories, California, USA), 300 μg/mouse, was injected intra-peritoneally (i.p.) immediately after the MOG injection and then 48 h later. Mice were examined daily. EAE was scored as follows: 0, no abnormality; 1, mild limp tail weakness (floppy tail); 2, tail paralysis; 3, tail paralysis and hind leg paresis; 4, hind leg paralysis or mild forelimb weakness; 5, quadriplegia or moribund state; 6, death.

### FTS Treatment

FTS treatment was administered by protocols that have been previously described (Aronovich et al., 2005; Kafri et al., 2005; Karussis et al., 2001; Katzav et al., 2003, 2001; Mor et al., 2008, 2009). Briefly, FTS (as powder) was diluted in chloroform (35.8 mg/ml of FTS=0.1 M) and kept in aliquots. The content (280 μl) of one aliquot was evaporated under nitrogen and then dissolved in 40 μl absolute ethanol and 15 μl NaOH; 2.5 ml of PBS was subsequently added. Each mouse received 0.1 ml of this solution daily (0.4 mg/mouse which on a per unit weight basis equates to 20 mg/kg) intraperitoneally (i.p.), starting from day 9 of disease induction, just before the clinical onset of paralysis.

### Glatiramer Acetate (GA) Treatment

GA (Copaxone®) (2 mg/mouse i.e. 100 mg/kg or 300 μg/mouse, i.e., 15 mg/kg) was given subcutaneously (s.c.), starting from day 9 after the induction, just before the clinical onset of paralysis, as described previously (Aharoni et al., 2005, 2008; Amon and Aharoni, 2009).

### MR Imaging of EAE Induced Mice

The treatment effect was evaluated by MRI experiments which were conducted on day 14 after EAE-induction. Vehicle, FTS− (20 mg/kg/day, i.p., daily) and GA− (15 mg/kg/day, s.c., daily) alone-treated and FTS (20 mg/kg/day, i.p., daily) plus GA (15 mg/kg/day, s.c., daily) combined treated mice were scanned (n=8 per group) at their lower limb plexuses (L1-S3) of the spinal cord, according to where the damage to blood-brain barrier (BBB) and myelin lesions are expected to be. During the MRI scanning, mice were anesthetized with isofluorane (3% for induction, 1-2% for maintenance) mixed with compressed air (1 l/min) delivered through a nasal mask. Once anesthetized, the animals were placed in a body-holder to assure reproducible positioning inside the magnet. Respiration rate was monitored and maintained throughout the experimental period at 60-80 breaths/min. MRI experiments were performed on a 7T Bruker scanner (70/30 USR Bruker BioSpec, Germany) equipped with a gradient coil system capable of producing pulse gradients of up to 400 mT/m in each of the three directions. All MR images were acquired to scan mice's spinal cord which was located on the surface coil and transmitter linear coil. Axial images of the lumbar part of the spinal cord have been taken. The MRI protocol included T₂ maps and T₁-weighted sequences before and after administration of 0.5 mmol/kg body weight Gd-DTPA. The T₂ map was acquired using the multi-slice multi-echo (MSME) spin-echo imaging sequence with the following parameters: a repetition delay (TR) of 3600 ms, 16 ms time echo (TE) increments (linearly from 10 to 160 ms), matrix dimension of 256×96 (interpolated to 256×256) and two averages, corresponding to an image acquisition time of 6 min 48 s. The T₂ data set consisted of 16 images per slice. Twenty continuous slices with a slice thickness of 0.8 mm were acquired with a field of view (FOV) of 25×15 mm².

The T₁-weighted images were acquired using the following parameters: a repetition delay (TR) of 1100 ms, 9.75 ms time echo (TE) increments, matrix dimension of 320×144 (interpolated to 320×122) and two averages, corresponding to an image acquisition time of 5 min 30 s. Twenty continuous slices with a slice thickness of 0.8 mm were acquired with a field of view (FOV) of 25×15 mm².

### MRI Analysis

T₂-map MRI was used to deliberate the EAE lesions (demyelination) in the mice's spinal cords. Lesion volume was calculated from the T₂-map MR images using the MATLAB® image processing toolbox. The analysis was performed by defining, manually, regions of interest (ROIs) corresponding to the lesion area in the spinal cord and to the parallel normal appearing area at the same slice. Area was considered as lesion area when it had higher intensity as compared to the parallel area at the same slice. Two types of analysis were done using T₂-map data. In the first analysis, for each mouse, the T₂ value of the higher intensity region in each slice (20 slices per mouse) was multiplied by the number of voxels in that region. These multiplies were summarized and divided by the sum of voxels per that mouse. From that value was then subtracted the value of a normal appearing parallel tissue in the same slice which was measured in the same way as described above. The calculation is described in the following equation:

$${T}_{2} = {( \frac{{\sum }_{i = 1}^{20} {( {T}_{2} \times {N}_{voxels} )}_{i}}{{\sum }_{i = 1}^{20} {N}_{voxels}} )}_{Lesion} - {( \frac{{\sum }_{i = 1}^{20} {( {T}_{2} \times {N}_{voxels} )}_{i}}{{\sum }_{i = 1}^{20} {N}_{voxels}} )}_{{Normal}_{α} {ppearing}_{i} issue}$$

In the second analysis of T₂ map, the volume (in mm³) of the enhanced region (lesions) were defined and accumulated per mouse.

The gadolinium enhancement obtained from T₁-weighted MRI reflects the infusate distribution in the mice's blood-brain barrier within the spinal cord. Thus, the volume (in mm³) of infusate distribution was calculated from the T₁-weighted MRI. Regions of interest (ROIs) were defined over the entire enhancing region in each slice using the MATLAB® image processing toolbox. The volume in the regions of interest was counted and accumulated for each mouse.

### Histology

To assess the pathological changes in the vehicle, FTS− (20 mg/kg/day, i.p., daily) and GA− (15 mg/kg/day, s.c., daily) treated mice and in FTS (20 mg/kg/day, i.p., daily) plus GA− (15 mg/kg/day, s.c., daily) treated mice, the mice were sacrificed on day 16 after EAE-induction. The lumbar part of the mice's spinal cord (n=8 mice for each group) was fixed in 4% paraformaldehyde (Electric Microscopy Science, PA, USA) and embedded in paraffin and cut in 4-μm-thick axial sections. Sections were stained with hematoxylin and eosin (H&E) and luxol fast blue (LFB) and then mounted in histomount medium (Invitrogen Corporation, CA, USA). Images were examined under a light microscope and captured with a NikonDS-5Mcamera (NikonInstech, Tokyo, Japan). Quantification of the inflammation and demyelination was conducted as previously described (Mi, et al., 2007). Briefly, to quantify inflammation H&E-stained nuclei were imaged, counted and percentage of stained cells per field were calculated. Similarly the percentage of regions with demyelination per field was estimated by counting the LFB-stained images. Imaging was aided by the use of Image-Pro Plus 5.1 software (MediaCybernetics, Silver, Spring, Md.).

### Western Blotting and Ras-GTPase Pull-Down Assay

On day 16 post EAE-induction, the brains and splenocytes of the mice (n=4 per each group) were homogenized and their lysates were subjected to sodium dodecyl sulfate-olyacrylamide gel electrophoresis followed by western blotting, as described (Goldberg and Kloog, 2006), with one of the following antibodies: pan-RAS (Ab-3; Calbiochem, San Diego, Calif.), anti-CD3 Ab (AbD serotec, Oxford, UK), anti-ERK (Santa Cruz Biotechnology, CA. USA), anti phospho-ERK (Sigma, USA), anti-tubulin Ab and anti-Foxp3 Ab (eBioScience, San Diego, Calif.). Protein bands were visualized by enhanced chemiluminescence (ECL) kit (Amersham Pharmacia Biotech, Arlington Heights, Ill.) and quantified by densitometry with Image EZQuant-Gel software (Copyright©2005, EZQuant Ltd). Lysates were used to determine Ras-GTP by the glutathione S-transferase (GST)-Ras-binding domain pulldown assay, and this was followed by western blotting with anti pan-Ras Ab.

### Proliferative Response of Lymphocytes

Pooled single cell suspension of spleens were obtained on day 17 post-EAE induction and assayed in vitro for their response to antigens and mitogens (Myelin basic protein—MOG, lipopolysacharide—LPS, and concanavalin A—ConA) by a proliferation assay. The assay was carried out by plating in each microculture well 2×10⁴ cells in 0.1 ml of proliferation medium containing optimal concentration of antigens as follows: 25 μg/ml MOG, 50 μg/ml MOG, 20 μg/ml LPS or 1 μg/ml of ConA. The experiments were performed in triplicate in 96-well, flat-bottom, microtiter plates (Costar, Cambridge, USA). Cultures were incubated for 72 h in humidified atmosphere of 95% air and 5% CO₂ at 37° C. Cell proliferation was determined by Colorimetric Bromodeoxyuridine (BrdU) Cell Proliferation kit (Calbiochem, Darmstadt, Germany) following manufacturer's instructions. In brief, 20 μl of BrdU labeling solution diluted 1:2000 in culture medium were added to each well for the last 18 h of culture. After removing the medium, cells were fixed, and anti-BrdU peroxidase working solution was added to each well and incubated for one (1) hour at room temperature. Following several washes, substrate solution was added, color was developed, and absorbance was measured with a microplate reader at 450 nm. The stimulation index (S.I.) was calculated as follows: the mean absorbance of cells culture in the presence of antigen divided by the mean absorbance of cells in the absence of antigen.

### ELISA for Cytokines

Levels of serum IL-10, IL-4, IFN-γ and IL-17 cytokines were determined on day 16 post EAE-induction using an ELISA kit for each of the cytokines (Bender MedSystem, Vienna, Austria).

### Statistical Analysis

Descriptive analysis data are presented as mean±S.E.M. Statistical analysis was carried out by using Student's t-test, one-way ANOVA, Fisher's exact test and Kruskal-Wallis non-parametric ANOVA based on ranks with a Dunn's multiple comparison test were used to compare the different experimental groups. P value<0.05 was considered significant.

### Results

### Combined Treatment of FTS and GA Suppressed Synergistically the Clinical Signs of EAE

EAE was induced in C57bl/6 mice with MOG and drug treatment started on day 9 of disease induction. The animals were divided into four (4) groups. Mice in one group received the combined treatment of FTS (20 mg/kg/day, i.p., daily) and GA (100 mg/kg/day, s.c., daily), mice in the second group received FTS (20 mg/kg/day, i.p., daily), mice in the third group received GA (100 mg/kg/day, s.c., daily) and mice in the fourth group received the vehicle only. In three separate experiments in which delayed treatment was performed, starting just before the onset of clinical signs of EAE (day 9), 23 of 29 (79.3%) vehicle-treated animals, 19 of 27 (70.3%) GA-treated animals and 17 of 28 (60.7%) FTS-treated animals developed clinical signs of EAE compared to 2 of 29 (6.89%) of combined treated (FTS and GA) mice (p<10⁻⁶ vs. control and GA or FTS alone treated mice (Fisher's exact test).

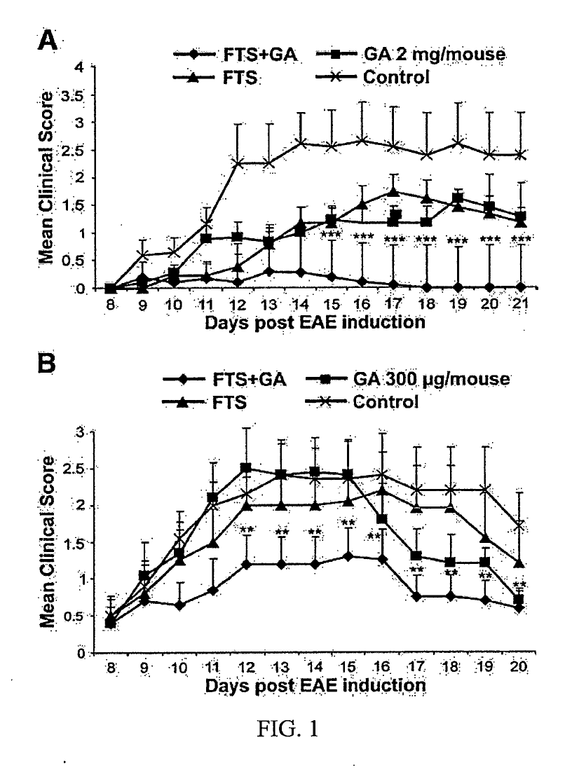

The clinical scores of the mice were recorded and shown in FIG. 1A. The maximal average score in the control sham-treated group was 2.52±0.66, in the GA-treated group 1.4±0.43 (p<0.01 vs. sham-treated, Kruskal-Wallis test), in the FTS-treated group 1.07±0.6 (p<0.01 vs. control, Kruskal-Wallis test), whereas in the combined-treatment group (FTS and GA) it was significantly lower (0.203±0.18, p<0.001 vs. control-treated, p<0.01 vs. GA-treated and p<0.01 vs. FTS-treated, Kruskal-Wallis test). Thus, the clinical course of EAE in the combined FTS and GA treatment group of mice was significantly ameliorated as compared to that in the sham-treated control group, GA− or FTS-alone-treated groups (p<0.0001, Kruskal-Wallis test, FIG. 1A).

To explore whether these results were associated with the maximally high GA dose used in some of the animal experiments (100 mg/kg/day) (Aharoni, et al., 2005), the experiment was repeated, using a dose of GA similar to the dosage used in human patients (15 mg/kg/day). In four different experiments (four groups of mice in each experiment as detailed above with n=10 in each group), it was found that 35 of 40 (87.5%) vehicle-treated animals, 31 of 40 (77.5%) GA-treated animals and 33 of 40 (82.5%) FTS-treated animals developed clinical signs of EAE compared to 9 of 40 (22.5%) of the combined-treatment mice (FTS and GA; p<0.001 vs. control and GA or FTS-treated mice; Fisher's exact test). The clinical scores of these experiments are shown in FIG. 1B. The maximal average score in the control sham-treated group was 2.4±0.5, in the GA-treated group 2.38±0.61 and in the FTS-treated group 2.15±0.45, whereas in the combined-treatment group (FTS and GA) it was significantly lower (1.35±0.3, p<0.01 vs. control-treated mice, Kruskal-Wallis test). A significant decrease in clinical signs due to combined treatment of FTS and GA (1.35±0.3) compared to control (2.4±0.5) or GA (2.38±0.61) and FTS (2.15±0.45) alone were recorded in this experiment (p<0.001 vs. control, p<0.05 vs. GA− or FTS-alone-treated mice, Kruskal-Wallis test) (FIG. 1B). The FTS-treated mice and the GA-treated mice yielded clinical scores of 0.25±0.11 (e.g., 2.4 minus 2.15) and 0.02±0.05 (e.g., 2.4 minus 2.38) respectively while FTS plus GA yielded a score of 1.05±0.2 which represents 43.7% of the maximal response ((1.05/2.4)×100=43.7%); namely, each of the compounds alone yielded scores of 0.02 (2%), and 0.25 (25%) for GA and FTS respectively. The sum of the individual treatments was then 25.2%, significantly lower than the score of the combined treatment (43.7%), indicative of synergism between GA and FTS.

Combined Treatment of FTS with GA Reduces the Lesions and Disruption of the Blood-Brain Barrier in the Spinal Cords of EAE Mice as Determined by MRI

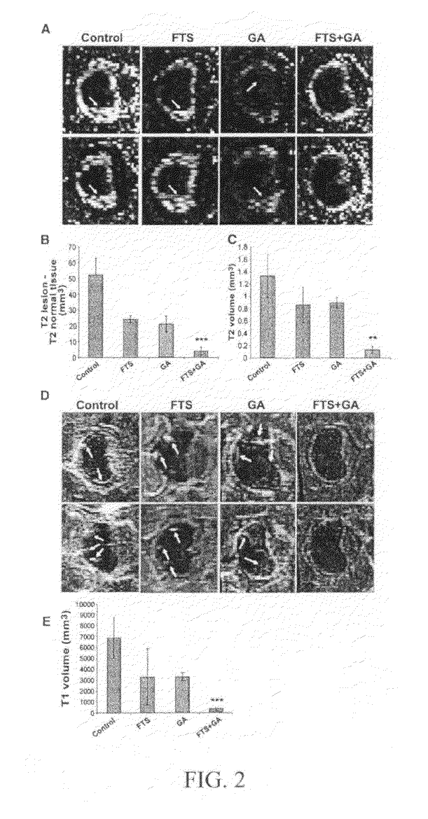

MR imaging was used to determine the possible effects of each of the three treatments on the pathological damage induced in the EAE model. Fourteen days post EAE-induction, MRI was performed on controls, FTS (20 mg/kg/day, i.p., daily), GA (15 mg/kg/day, s.c., daily) and FTS (20 mg/kg/day, i.p., daily) plus GA− (15 mg/kg/day, s.c., daily) treated groups (n=8 per group) (FIG. 2A). MRI was performed at the lower part of the spinal cord (L1-S3) as described above. The MRI protocol included T₂-maps and T₁-weighted sequences before and after administration of Gd-DTPA. T₂-map images may imitate a variety of pathological processes and EAE conditions such as focal lesions (demyelination), lymphocyte inflammation, edema, axon loss and gliosis (Weerth, et al., 2003). Analysis of T₂-map sections demonstrated a significant decrease in focal lesions among the FTS and GA combined treated mice. This was apparent when the value of the normal area was subtracted from the value of the lesion at the same slice (92.7%±13.25% decrease vs. control, p<0.01, Kruskal-Wallis test) (FIG. 2B) and when the total volume of lesions per mouse was measured and accumulated (89.78%±11.6% decrease vs. control, p<0.01, Kruskal-Wallis test) (FIG. 2C). In the later analysis where total volume of lesions per mouse was measured (FIG. 2C) a clear synergistic effect is demonstrated: While the individual treatment yielded only 54.15%±13% reduction (30.1%±7% for FTS+ and 24.05%±6% for GA), the yield of the combined treatment was significantly higher (89.78%±11.6%).

Finally the T₁-weighted MRI in the four groups was determined using Gd-DTPA enhancement which is suggestive of disruption of the blood-brain barrier due to inflammatory demyelination (Noseworthy, et al., 2000). Analysis of the T₁-weighted images showed a significant decrease in the Gd-DTPA enhancement within the FTS and GA combined treated mice compared to the controls (decrease of 95.1%±10.2%, p<0.001, Kruskal-Wallis test) (FIGS. 2D and E).

Combined Treatment of FTS with GA Reduces the Infiltration and Demyelination in the Spinal Cords of EAE Mice

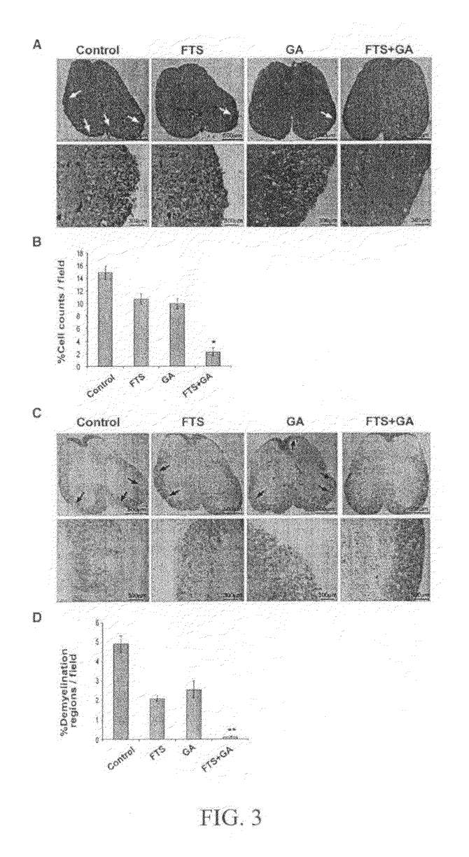

In order to establish the cellular infiltration and the pathological damage induced in the EAE mice, control mice, and mice treated with FTS (20 mg/kg/day, i.p., daily), GA (15 mg/kg/day, s.c., daily) or FTS (20 mg/kg/day, i.p., daily) plus GA (15 mg/kg/day, s.c., daily) were sacrificed on day 16 post EAE-induction and the lumbar part of their spinal cords was removed and stained with H&E and LFB reagents (n=8 per each group). Analysis of nuclei counts demonstrated a significant reduction of regions with inflammatory infiltrates in spinal cords of the combined-treatment mice compared to control mice (decrease of 84.72%±21%, p<0.05, Kruskal-Wallis test) whereas only a 33.12%±6.98% and 28.44%±11.2% reduction was observed in spinal cords of mice treated with GA or FTS respectively (FIGS. 3A, B).

Thus the combined treatment caused a reduction of 84.72%±21% while the effect of the individual treatment caused a reduction of 61.56%±18.18% ((33.12%±6.98%)+(28.44%±11.2%)), suggesting a synergistic effect. Similar results were observed when an analysis of demyelination was performed. A significant decrease of 94.5%±16% in regions of demyelination was found in the spinal cords of the combined-treatment group of mice compared to control mice. No significant decrease was detected using only one of the therapies (reduction of 48.97%±6.78% and 59.18±10.6% with GA and FTS, respectively) (FIGS. 3C, D). Both infiltration and demyelination were mostly detected within the peripheral parts of the axial section of the spinal cord (p<0.01, Kruskal-Wallis test).

### Combined Treatment of FTS and GA In Vivo Induced Increased Foxp3 Levels and Reduced the Amount RAS, RAS-GTP and P-ERK in Splenocytes

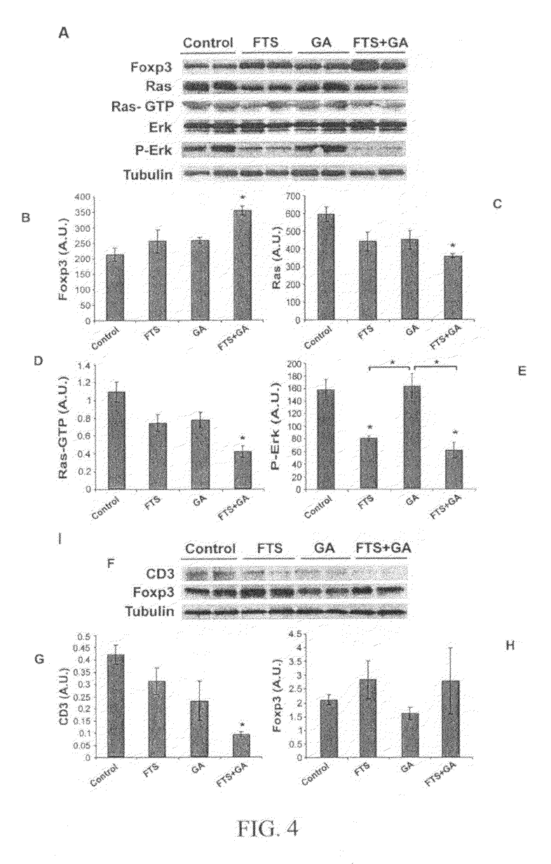

Splenocytes from control, FTS− (20 mg/kg/day, i.p., daily) or GA− (15 mg/kg/day, s.c., daily) alone-treated and of FTS (20 mg/kg/day, i.p., daily) plus GA− (15 mg/kg/day, s.c., daily) treated mice were obtained on day 17 post EAE-induction. The organs were homogenized and the amount of Foxp3, total Ras, Ras-GTP, Erk and P-Erk were determined by western immunoblotting using specific antibodies (see Materials and Methods). The results (FIGS. 4A and B) demonstrate a significant increase of the Foxp3 level within the splenocytes of FTS and GA combined treated mice compared to control mice (increase of 167.4%±14%, p<0.05, ANOVA). No significant elevation was found when the treatment was with each of the drugs alone (FIGS. 4A and B).

The next set of experiments was designed to understand the molecular mechanisms that are involved in the synergistic effects of FTS and GA on EAE. The impact of the various treatments on the levels of Ras, Ras-GTP and phospho-Erk was examined. It was found that the levels of Ras and Ras-GTP were significantly decreased as a result of the combined treatment of FTS plus GA compared to control-treated mice (40%±5% and 19.76%±4.3% decrease in Ras and Ras-GTP levels, respectively, p<0.05, ANOVA). The levels of phospho-Erk were also decreased following the combined treatment of FTS and GA (62.04%±6% and 61.1%±8.3% vs. GA alone and control-treated mice, respectively, p<0.05, ANOVA) (FIGS. 4A, C and D). However, it was found that phospho-Erk levels were also decreased by FTS treatment alone (51.38%±5.6% and 50.18%±9.1% vs. GA alone and control mice, respectively, p<0.05, ANOVA) (FIGS. 4A and E). No significant change was detected regarding total Erk levels.

These results suggest that the main effect of FTS is to reduce the levels of Ras-GTP and of activation of its downstream target ERK as well as the relief of Ras-GTP inhibition of Foxp3 expression. GA on the other hand does not reduce significantly Ras or phospho-ERK and does not elevate Foxp3 (FIGS. 4A, C, E and B). Thus GA acts through a mechanism that is distinct of that of FTS which acts on Ras-GTP. The distinct mechanisms through which GA and FTS act to elevate EAE explain their synergism.

### The Combined Treatment of FTS and GA In Vivo Reduced the Amount of CD3 Lymphocytes Infiltrating the EAE Brain

In order to determine the amount of lymphocytes that infiltrate the brains of EAE-induced mice brains from control, FTS− (20 mg/kg/day, i.p., daily) or GA− (15 mg/kg/day, s.c., daily) alone-treated and of FTS (20 mg/kg/day, i.p., daily) plus GA− (15 mg/kg/day, s.c., daily) treated mice were obtained on day 17 post EAE-induction (n=4 per each group). Brains of the mice were then homogenized and tested by western immunoblotting for levels of CD3, a membrane marker for lymphocytes and for Foxp3 levels.

The results of these experiments (FIGS. 4F, G and H) demonstrated a significant decrease in CD3 levels within the brains of FTS and GA combined treated mice compared to control mice (decrease of 77.6%±9.6%, p<0.05,ANOVA). No significant change in the level of Foxp3 within the brain was detected. These data indicate that the combined treatment of FTS and GA prevents lymphocytes from infiltrating into the brain to induce inflammatory demyelination.

### Specific and Synergistic Suppression of the Lymphocytic Proliferative Responses to Myelin Antigen (MOG) by Combined Treatment of FTS and GA in EAE

Lymphocytes were obtained from the spleens of mice on day 16 post immunization with MOG and subjected to ex vivo BrdU incorporation proliferation assays. The lymphocytes were obtained from spleens of mice (n=8 per each group) treated with FTS (20 mg/kg/day, i.p., daily) plus GA (15 mg/kg/day, s.c., daily), or from mice treated with FTS alone (20 mg/kg/day, i.p., daily), GA alone (15 mg/kg/day, s.c., daily) or from vehicle-treated mice, and then subjected to ex vivo BrdU incorporation proliferation assay. The cells, obtained from the four groups described above, were stimulated with various mitogens ex vivo for 48 h.

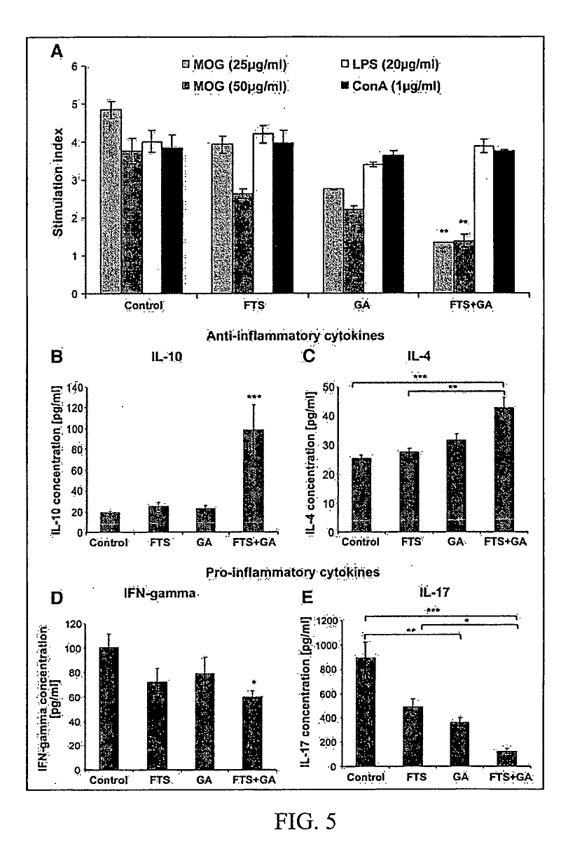

The results of these experiments demonstrated a modest decrease in the reactivity of lymphocytes to myelin antigen. Lymphocytes obtained from FTS-treated mice and stimulated with 25 μg/ml and 50 μg/ml MOG resulted in 17.85%±5.34% and 29.9%±9.18% decrease in their proliferation, respectively as compared to lymphocytes obtained from control mice (FIG. 5A). Accordingly, lymphocytes obtained from GA-treated mice and stimulated with the indicated concentrations of MOG resulted in 43.19%±6.35% and 41.22%±6.12% decrease in their proliferation, respectively as compared to control mice (FIG. 5A). Lymphocytes of the combined treatment exhibited a far lower response to 25 μg/ml and 50 μg/ml MOG as compared with the lymphocytes of the control mice (72.41%±13.6% and 63.51%±9.87% decrease, respectively, FIG. 5A), indicating a robust suppression.

Thus the combined treatment yielded a reduction of 72.41%±13.6% in proliferation as response to 25 μg/ml MOG while the effect of the individual treatments yielded 61.04%±11.69% (17.85%±5.34% plus 43.19%±6.35%) reduction, demonstrating a synergistic effect. No significant differences were found between lymphocytes of control and treated mice in the response to LPS and ConA (FIG. 5A). Taken together, these results of the ex vivo experiments demonstrated that the combined treatment of FTS and GA inhibited, synergistically and selectively, those lymphocytes that respond to the sensitizing antigen MOG.

The Combined Treatment with FTS and GA In Vivo Increased the Amount of Anti-Inflammatory Cytokines and Decreased the Amount of Pro-Inflammatory Cytokines

To delineate the effect of the combined treatment with FTS and GA at a cellular level, the levels of cytokines in the serum of treated mice (8 per group) were determined. Serum was obtained from FTS (20 mg/kg/day, i.p., daily) plus GA− (15 mg/kg/day, s.c., daily), treated mice or from mice treated with FTS alone (20 mg/kg/day, i.p., daily), GA alone (15 mg/kg/day, s.c., daily) and from vehicle-treated mice (n=10 mice per group). The sera were tested for their cytokine levels. As shown in FIGS. 5B-E, the combined treatment of FTS and GA resulted in a significantly increased level of anti-inflammatory cytokines (514.2%±23.5% and 175.89%±24.98% in serum IL-10 and IL-4 levels, respectively, p<0.001, Kruskal-Wallis test) and a decreased level of pro-inflammatory cytokines (i.e., 40.2%±12.36% and 85.97±18.69% decrease in serum IFN-γ and IL-17 levels respectively, p<0.05 and p<0.001, Kruskal-Wallis test). In addition, serum obtained from FTS plus GA-treated mice demonstrated a significant increase in IL-4 levels (155.3%±23.62% increase, p<0.01, Kruskal-Wallis test) and a decrease in IL-17 levels (68.42%±11.4 decrease, p<0.05, Kruskal-Wallis test) compared to FTS-treated mice (FIGS. 5C and E). Furthermore, GA alone caused significant decrease in IL-17 level compared to control sham-treated mice (74.44%±6.57% decrease, p<0.01, Kruskal-Wallis test) (FIG. 5E).

### CONCLUSIONS

The results presented here indicate a synergistic beneficial effect of the combined FTS and GA treatment of EAE in contrast to treatment with either agent alone.

The additive effect of FTS together with GA was detected in the clinical disability of the animals when the dose of GA was similar to the dose when GA is taken alone. When a clinically relevant dose of GA was administrated, a clear synergistic effect was observed. These results were accompanied by a significant reduction in EAE focal lesions and demyelination within the lumbar part of the spinal cord and a decrease in detection of the blood-brain barrier breakdown which is substantiated by the finding of less inflammatory CNS infiltration and specifically smaller numbers of lymphocytes. Focal lesions of EAE disease and blood-brain barrier breakdown were is limited due to the small size of the spinal cord. The synergistic immunological effect of FTS together with GA appears especially specific for the MOG antigen used to generate the disease in the animal. Moreover, observation of the effect of combined therapy at the cellular level revealed a significant increase in Th2 anti-inflammatory cytokines (IL-4 and IL-10) in the mice sera while at the same time a reduction in Th1 pro-inflammatory cytokines (IFN-γ and IL-17).

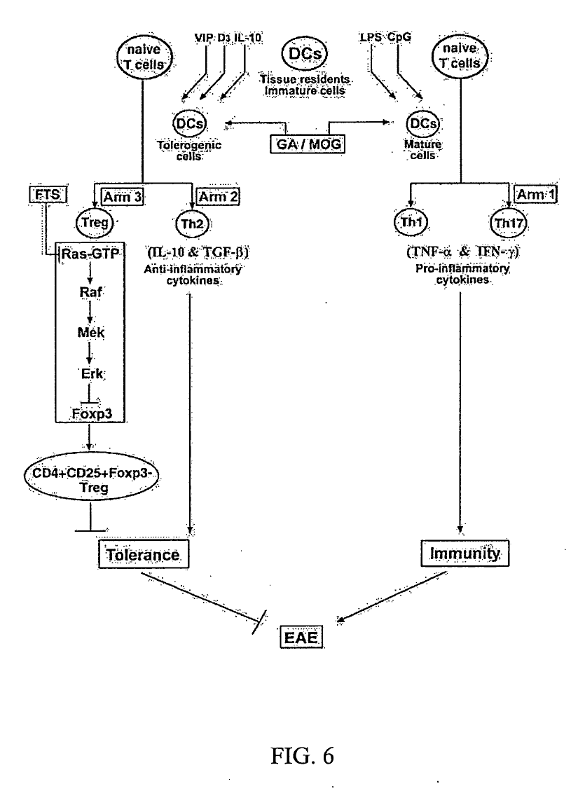

While not intending to be bound by any particular theory of operation, Applicants hypothesize that the synergistic effect of FTS and GA can be explained at least in part their distinct mechanism of action (see Scheme in FIG. 6). FTS is a Ras inhibitor that acts in a rather specific manner on the active GTP-bound form of Ras. It inhibits GTP-bound forms of H-, N-, and K-Ras proteins (Gana-Weisz, et al., 2002; Weisz, et al., 1999) (see also Arm 3 in FIG. 6). More specifically, FTS competes with Ras-GTP for binding to specific saturable binding sites in the plasma membrane, resulting in mislocalization of active Ras and facilitating Ras degradation (Haklai, et al., 1998). FTS disrupts the interactions of H-Ras-GTP and its chaperon galectin-1 and of K-Ras-GTP and its chaperon galectin-3 (Belanis, et al., 2008; Shalom-Feuerstein, et al., 2008). Disruptions of these interactions by FTS induce Ras mislocalization (Rotblat, et al., 2008). This competitive inhibition prevents active Ras from interacting with its prominent downstream effectors and results in reversal of the transformed phenotype in transformed cells that harbor activated Ras. As a consequence, Ras-dependent cell growth and transforming activities, both ex vivo and in vivo, are strongly inhibited by FTS (Gana-Weisz, et al., 2002; Weisz, et al., 1999).

The present results show that although the combined treatment of FTS and GA decreases the levels of Ras and Ras-GTP, the decrease in phospho-Erk protein was achieved largely due to FTS treatment alone. These results suggest that FTS alone is sufficient to strongly down regulate lymphocyte proliferation through Ras inhibition and consequently prevent the initial inflammatory damage inflicted on the myelin by the lymphocytes. Furthermore, FTS has an important immunoregulatory effect. We examined the levels of Foxp3 within the lymphocytes and found a significant synergistic elevation within the FTS plus GA combined-treatment mice as compared to control-treated mice. This elevation in Foxp3 levels was not detected in the brains of the combined treated mice, results that correspond with the observed decrease in CD3 lymphocytes within the brains of the combined treated mice. Taken together, these data indicate that the immunosuppressive effect of Foxp3+ Tregs is being conducted within the peripheral organs, such as spleen and lymph nodes. This effect prevents active inflammatory T cells from infiltrating the brain, and therefore preserves the myelin sheaths from being damaged.

The unexpected strong synergistic effect of GA and FTS is explained by distinct molecular mechanisms. FTS provides its beneficial protective effects by inhibiting active Ras and its signal to ERK and to Foxp3 while GA has it own effects on the anti-inflammatory T-helper type 2 (Th2) cells which are not thought to depend on Ras (Vieira, et al., 2003). The proposed model depicted in FIG. 6 is based on Applicants present results taken together with previous studies on tolerance and immunity, on the impacts of MOG immunization, and on the effects of GA and FTS on EAE. Accordingly, tissue resident immature dendritic cells (DCs) are induced to differentiate by factors of inflammation and immunity such as LPS or CpG or other toxins to mature DCs which then serve as antigen-presenting-cells (APCs). The APCs interact with antigens including MOG and induce the differentiation of naive T cells into Th1 cells or Th17 cells which respectively produce the pro-inflammatory cytokines such as TNF-α and IFN-γ (Murphy et al.) (see also Arm 1 in FIG. 6). This is a significant part of the immunity induced by the MOG antigen in EAE (Murphy et al.).

Immature DCs are also affected by tolerogenic factors such as VIP, D3 or IL-10, as well as by GA (Auray et al.; Chorny, et al., 2005; Wakkach, et al., 2003) (see also FIG. 6) which convert them to tolerogenic DCs (FIG. 6, Arm 2). These DCs acting as APCs convert immature T cells into Th2 cells that produce the anti-inflammatory cytokines IL-10 and TGF-13 (FIG. 6 Arm 2). Previous studies have demonstrated that the immunoregulatory effect of GA is achieved through its interactions with tolerogenic DCs which induce Th2 cells (Vieira, et al., 2003) (Arm 2 in FIG. 6). Hence, the tolerance accomplished by GA, et al., inhibition in the EAE symptoms is carried out by a mechanism that is not known to depend on Ras (Vieira, et al., 2003) (see also FIG. 6, Arm 2). This mechanism is distinct from a third mechanism (FIG. 6, Arm 3) whereby the Ras-GTP signal blocks Foxp3 transcription in CD4+CD25+ Tregs (Mor, et al., 2008, 2009) (see also scheme in FIG. 6 Arm 3). Once Ras signaling is blocked by FTS the Ras-dependent block of Foxp3 transcription is relieved and the newly formed CD4+CD25+Foxp3+ Tregs promote immune tolerance (see also Scheme in FIG. 5). Thus FTS and GA operate on two distinct mechanisms leading to the synergistic anti-inflammatory block in EAE (Scheme in FIG. 6).

### Example II

### Combined Treatment of FTS Administered Per Os with GA Suppressed the Clinical Signs of EAE

EAE was induced in C57bl/6 mice with MOG and drug treatment started on day 9 after disease induction (see Materials and Methods) and mice in one group received the combined treatment of FTS (60 mg/kg/day, p.o., daily) and GA (15 mg/kg/day, s.c, daily), mice in the second group received FTS (60 mg/kg/day, p.o., daily), mice in the third group received GA (15 mg/kg/day, s.c., daily) and mice in the fourth group received the vehicle only. In the delayed treatment, starting just before the onset of clinical signs of EAE (day 9), we found that 9 of 10 (90%) vehicle-treated animals, 7 of 10 (70%) GA treated animals and 6 of 10 (60%) FTS treated animals developed clinical signs of EAE compared to 3 of 10 (30%) of the combined treatment mice (FTS and GA) mice (p<10⁻³ vs. control and GA or FTS− alone treated mice, Fisher's exact test).

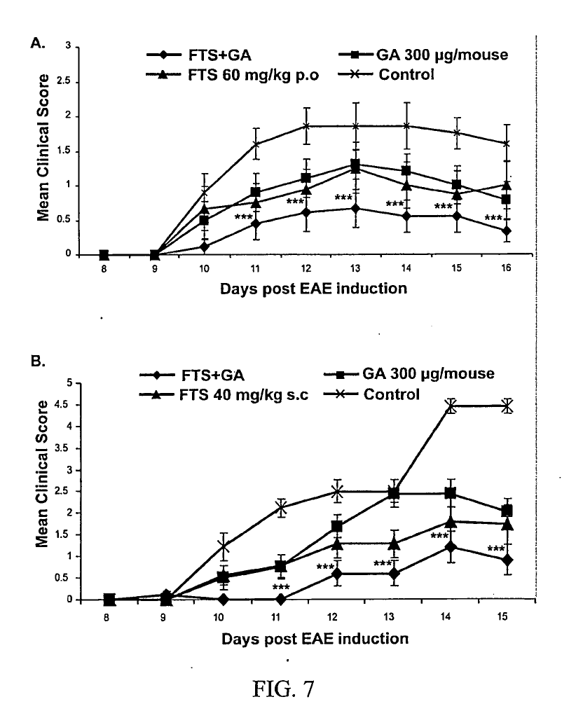

The clinical scores of the mice recorded and shown in FIG. 7A. The maximal average score in the control sham treated group was 1.85±0.25, in the GA-treated group 1.3±0.213 (p<0.01 vs. sham-treated, Kruskal-Wallis test), in the FTS-treated group 1±0.33 (p<0.01 vs. control, Kruskal-Wallis test), whereas in the combined treatment group (FTS and GA) it was significantly lower (0.66±0.27, p<0.001 vs. control-treated, p<0.01 vs. GA-treated and p<0.01 vs. FTS-treated, Kruskal-Wallis test). Thus, the clinical course of EAE in the combined FTS (administrated per os) and GA (administrated subcutaneous) treatment group of mice was significantly ameliorated as compared to that in the sham-treated control group, GA or FTS alone treated groups (p<0.001, Kruskal-Wallis test, FIG. 7A).

### Example III

### Combined Treatment of FTS Administrated Subcutaneous with GA Suppressed the Clinical Signs of EAE

In our next experiment we examined whether the formulation of FTS dissolved in GA might be effective for treatment for EAE. For that matter, EAE was induced in C57bl/6 mice with MOG and drug treatment started on day 9 after disease induction (see Materials and Methods) and mice in one group received the combined treatment of FTS (40 mg/kg/day, s.c., daily) and GA (15 mg/kg/day, s.c, daily), mice in the second group received FTS (40 mg/kg/day, s.c., daily), mice in the third group received GA (15 mg/kg/day, s.c., daily) and mice in the fourth group received the vehicle only. In the delayed treatment, starting just before the onset of clinical signs of EAE (day 9), we found that 10 of 10 (100%) vehicle-treated animals, 9 of 10 (90%) GA treated animals and 9 of 10 (90%) FTS treated animals developed clinical signs of EAE compared to 5 of 10 (50%) of the combined treatment mice (FTS and GA) mice (p<10⁻³ vs. control and GA or FTS− alone treated mice, Fisher's exact test).

The clinical scores of the mice recorded and shown in FIG. 7B. The maximal average score in the control sham treated group was 4.44±0.16, in the GA-treated group 2.43±0.316 (p<0.01 vs. sham-treated, Kruskal-Wallis test), in the FTS-treated group 1.72±0.45 (p<0.01 vs. control, Kruskal-Wallis test), whereas in the combined treatment group (FTS and GA) it was significantly lower (1.2±0.35, p<0.001 vs. control-treated, p<0.01 vs. GA-treated and p<0.01 vs. FTS-treated, Kruskal-Wallis test). Thus, the clinical course of EAE in the combined FTS (dissolved in GA and administrated s.c.) and GA (administrated s.c.) treatment group of mice was significantly ameliorated as compared to that in the sham-treated control group, GA or FTS alone treated groups (p<0.001, Kruskal-Wallis test, FIG. 7B).

### Example IV

### Oral Dosage Forms Containing FTS and Laqunimod

A. Tablets Containing FTS (200 mg) and Laquinimod (0.2 mg)

FTS active pharmaceutical ingredient (2000 g), laquinimod active pharmaceutical ingredient (20 g), microcrystalline cellulose (2000 g), hypromellose (12 g), croscarmellose sodium (15 g), sodium acetate (50 g), and magnesium stearate (3 g) are blended to uniformity and compressed into tablets weighing 410 mg. Assuming a 5% loss on material transfers and tablet press start-up, adjustment, and shut-down, approximately 9,500 tablets containing 200 mg FTS and 0.2 mg laquinimod are yielded.

B. Capsules Containing FTS (200 mg) and Laquinimod (0.1 mg)

FTs active pharmaceutical ingredient (1500 g), laquinimod active pharmaceutical ingredient (7.5 g), microcrystalline cellulose (200 g), sodium acetate (20 g) and magnesium stearate (2 g) are blended to uniformity and filled into hard gelatin capsules. Assuming a 5% loss on material transfers and encapsulating machine start-up, adjustment, and shut-down, approximately 7,125 capsules containing 200 mg FTS and 0.1 mg laquinimod are yielded.

### CITATIONS OF REFERENCED PUBLICATIONS

Aharoni, et al., 2005. J. Neurosci. 25, 8217-8228.
Aharoni, et al., 2008. Proc. Natl. Acad. Sci. USA 105, 11358-11363.
Amon, et al., 2009. J. Neural Transm. 116, 1443-1449.
Aronovich, et al., 2005. Immunobiology 210, 781-788.
Auray, et al., Differential activation and maturation of two porcine DC populations following TLR ligand stimulation. Molecular Immunology. 47, 2103-2111.
Belanis, et al., 2008. Mol. Biol. Cell 19, 1404-1414.
Ben-Nun, et al., 1996. J. Neurol. 243, S14-S22.
Bertolotto, et al., 1999. J. Interferon Cytokine Res. 19, 575-581.
Chorny, et al., 2005. Proc. Natl. Acad. Sci. USA 102, 13562-13567.
Gana-Weisz, et al., 2002. Clin. Cancer Res. 8, 555-565.
Goldberg, et al., 2006. Cancer Res. 66, 11709-11717.
Goodin, et al., 2002. Neurology 58, 169-178.
Haklai, et al., 1998. Biochemistry 37, 1306-1314.
Hogh, et al., 2000. Mult. Scler. 6, 226-230.
Isaksson, et al., 2009. Eur. J. Immunol. 39, 2925-2935.
Kafri, et al., 2005. J. Neuroimmunol. 168, 46-55.
Kalinski, et al., 1999. Immunol. Today 20, 561-567.
Karussis, et al., 2001. J. Neuroimmunol. 120, 1-9.
Katzav, et al., 2001. Clin. Exp. Immunol. 126, 570-577.
Katzav, et al., 2003. Immunobiology 207, 47-50.
Killestein, et al., 2001. Neurology 57, 1129-71131.
Li, et al., 2005. Blood 106, 3068-3073.
McDonald, et al., 2001. Ann. Neurol. 50, 121-127.
Menge, et al., 2008. Drugs 68, 2445-2468.
Mi, et al., 2007. Nat. Med. 13, 1228-1233.
Mor, et al., 2008. Eur. J. Immunol. 38, 1493-1502.
Mor, et al., 2009. Eur. J. Pharmacol. 616, 301-305.
Murphy, et al., 2010. Brain Behav Immun 24, 641-651.
Noseworthy, et al., 2000. N Engl J. Med. 343, 938-952.
Reis e Sousa, C., 2006. Nat. Rev. Immunol. 6, 476-483.
Rotblat, et al., 2008. Methods Enzymol. 439, 467-489.
Schildknecht, et al., 2010. Proceedings of the National Academy of Sciences of the United States of America 107, 199-203.
Shalom-Feuerstein, et al., 2008. Cancer Res. 68, 6608-6616.
Trapp, et al., 1998. N Engl J. Med. 338, 278-285.
Vieira, et al., 2003. J. Immunol. 170, 4483-4488.
Wakkach, et al., 2003. Immunity 18, 605-617.
Weerth, et al., 2003. Am. J. Pathol. 163, 1069-1080.
Weisz, et al., 1999. Oncogene 18, 2579-2588.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1-6. (canceled)

7. A method for treating a multiple sclerosis patient, comprising co-administering to the patient therapeutically effective amounts of a Ras antagonist represented by the formula 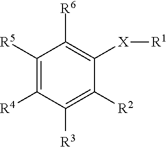
wherein X represents S; wherein R¹ represents farnesyl, or geranyl-geranyl; R² is COOR⁷, CONR⁷R⁸, or COOCHR⁹OR¹⁰, wherein R⁷ and R⁸ are each independently hydrogen, alkyl, or alkenyl; wherein R⁹ represents H or alkyl; and wherein R¹⁰ represents alkyl; and wherein R³, R⁴, R⁵ and R⁶ are each independently hydrogen, alkyl, alkenyl, alkoxy, halo, trifluoromethyl, trifluoromethoxy, or alkylmercapto; and
glatiramer acetate.

8. The method of claim 7, wherein the Ras antagonist and glatiramer acetate are administered in separate dosage forms.

9. The method of claim 7, wherein the Ras antagonist is administered via oral delivery.

10. The method of claim 9, wherein the Ras antagonist, is formulated in a tablet or capsule.

11. The method of claim 7, wherein the glatiramer acetate is administered subcutaneously.

12. The method of claim 7, wherein the Ras antagonist is FTS.

13. The method of claim 7, wherein the therapeutically effective amount of the Ras antagonist is about 400 mg to about 1200 mg per day.

14. The method of claim 7, wherein the therapeutically effective amount of the glatiramer acetate is about 20 mg/day.

15. The method of claim 7, wherein the Ras antagonist is an FTS analog selected from the group consisting of 5-chloro-FTS, 5-fluoro-FTS, FTS-methyl ester, FTS-amide, FTS-methylamide, FTS-dimethylamide, methoxymethyl S-farnesylthiosalicylate, methoxymethyl S-geranylgeranylthiosalicylate, methoxymethyl 5-fluoro-S-farnesylthiosalicylate, and ethoxymethyl S-farnesylthiosalicyate.

16. A kit for use in treating multiple sclerosis, comprising a first dosage form containing therapeutically effective amounts of the Ras antagonist as defined by the formula in claim 7, and glatiramer acetate, or separate dosage forms containing the Ras antagonist and glatiramer acetate, and optionally, written instructions for using the dosage form(s) to treat a multiple sclerosis patient.

17. The kit of claim 16, wherein the Ras antagonist is present in a first oral dosage form which is a tablet or capsule.

18. The kit of claim 16, wherein the glatiramer acetate is present in the form of a solution for injection.

19. The kit of claim 16, wherein the Ras antagonist is FTS.
